(19) United States
(12) Patent Application Publication
(10) Pub. No.: US 2009/0024116 A1
(43) Pub. Date: Jan. 22, 2009

(54) LASER HAIR AND SCALP TREATMENT DEVICE

(75) Inventors: Paul MULHAUSER, New York, NY (US); Lyndon T. TREACY, Long Island City, NY (US); Karl D. KIRK, III, New York, NY (US); Mark KRESS, Westport, CT (US)

Correspondence Address: STEFAN KIRCHANSKI, VENABLE LLP 2049 CENTURY PARK EAST, 21ST FLOOR, LOS ANGELES, CA, 90067, US

(73) Assignee: SPENCER FORREST, INC., Westport, CT (US)

(21) Appl. No.: 12/196,510

(22) Filed: Aug. 22, 2008

Related U.S. Application Data
(63) Continuation-in-part of application No. PCT/US2007/062593, filed on Feb. 22, 2007.
(60) Provisional application No. 60/804,167, filed on Jun. 7, 2006.
(60) Provisional application No. 60/776,907, filed on Feb. 22, 2006.

(51) Int. Cl. A61B 18/20

(52) U.S. Cl. 606/9

(57) ABSTRACT

A handled laser treatment device provides a plurality of branched light pipes or light distribution tips distributed so as to evenly deliver laser radiation to the surface of the scalp. To make efficient use of each laser diode, each laser can coupled to a branched light pipe with the branches arranged to distribute light directly to the surface of the scalp. Each laser source is connected to a three branch light pipe. Alternatively, each laser diode can be directly coupled to a light distribution tip arranged in a triad. Each light pipe or light distribution tip acts as a tine or tooth in a comb to bring laser radiation to the scalp free of obstruction by hair, etc., with a tripod configuration that allows each light pipe assembly to evenly contact the rounded surface of the scalp. The branched light pipes or the light distribution tips are preferentially coupled to a flexible membrane so as to be capable of independent orientation allowing improved contact of the distal ends of the tripods or triads to the scalp.

### CROSS-REFERENCE TO PRIOR APPLICATIONS

The present application is a continuation-in-part of and claims priority from copending PCT/US2007/062593, filed Feb. 22, 2007, which designated the United States and which in turn was based on and claimed priority from U.S. Provisional Patent Application No. 60/776,907 filed Feb. 22, 2006 and from U.S. Provisional Patent Application No. 60/804,167 filed Jun. 7, 2006, the contents of all these applications being incorporated by reference herein.

### U.S. GOVERNMENT SUPPORT

NA

### BACKGROUND OF THE INVENTION

1. Area of the Art

The current invention is in the area of topical scalp treatment with electromagnetic radiation and more particularly to a hand held device that delivers laser light to the scalp in an amount and distribution to stimulate hair growth.

2. Description of the Background Art

Scalp hair in humans serves a number of important biological functions probably the most important being protection of the skull from blows and from sunburn. Yet humans of both sexes have long been enthralled by scalp hair to an extent far beyond its apparent biological importance. It is generally considered an important asset in the appearance of both males and females. Vast amounts of money are spent on cleaning, coloring and styling hair. When a person loses hair either due to disease or natural biological processes like male pattern baldness, that person will often go to great lengths to overcome the loss. Folk medicine is filled with numerous baldness cures based on herbs and various more or less bizarre procedures. Wigs and hairpieces have been used for thousands of years. A wide variety of cosmetic procedures are available to minimize the appearance of hair loss. Yet only in relatively recent time have methods been developed to actually return growing hair to the scalp.

Probably the oldest of these methods is surgical hair replacement. This technique takes living hair follicles from a region that has hair and transplants them into a region that lacks hair. Surgical methods are expensive and painful and may involve side effects such as infection. In addition, many persons lack adequate areas of normal hair growth so as to provide sufficient follicles to repopulate the scalp. As a result pharmaceutical means to promote hair growth have been developed. Probably the first was the topical application of minoxidil. While pharmaceutical approaches show some effectiveness, they are generally unable to reverse a case of profound baldness. In addition, both topical and systemic pharmaceutical agents can provoke a number of different side effects.

More recently it has been discovered that carefully targeted light treatments may positively effect hair growth. Generally the most effective treatments have involved application of laser light at red and far red wavelengths. The precise reason for efficacy is unknown and is it not certain that laser radiation is required. Most likely the laser sources are simply more efficient at delivering effective doses of light. The levels of effective laser radiation are relatively low, but it has been found that it is important to deliver sufficient radiation evenly to the scalp. Because lasers were originally rather bulky optical devices early units intended to accomplish such treatments incorporated cumbersome helmets and tended to be large units resembling X-ray machines or other instruments common in a medical setting. With the advent of solid state (diode) lasers it became possible to reduce the treatment device into a more or less hand held unit. An example of an early version of such a device can be found in U.S. Pat. No. 6,497,719 to Pearl et al. In that disclosure the laser source is embedded within a handheld comb or brush unit designed so that the tines or bristles part the hair to allow the laser light to bathe the surface of the scalp.

A drawback of devices that mimic combs or brushes has been an inability to deliver a consistent, evenly distributed and reproducible level of laser radiation to the surface of the scalp. One approach is to have one or more laser sources (such as laser diodes) within the body of the device and to deliver the laser light through an optical system usually consisting of lenses and/or reflectors. The laser radiation can be blocked by hairs on the scalp although teeth (as in a comb) or bristles (as in a brush) can be provided to selectively reveal portions of the scalp to the laser beam. Nevertheless, much laser radiation may still be blocked by hair. German Patent No. 9102407 to Mink discloses an improvement over typical teeth or bristles by coupling each laser diode to a light pipe which also acted as a tooth in a comb-like structure. Since the end of a comb tooth directly contacts the scalp, this device was able to deliver radiation directly to the surface of the scalp. However, it is difficult to move such a comb-like device evenly over the scalp surface to ensure even treatment. Furthermore, the tines of a comb are generally arranged in a straight line whereas the scalp is curved. Therefore, it is difficult or impossible for all of the laser light delivering tines to simultaneously contact the scalp.

The current invention is a handheld device that utilizes either straight or branched light pipes so that a plurality of points on the scalp can be irradiated at one time and because the points can be distributed over a significant area a relatively large region of the scalp can be evenly irradiated at one time. So that efficient contact can be made with the variably curved surface of the scalp, the points are preferably arranged in a tripod or triad configuration and embedded in a flexible matrix or membrane so that each tripod or triad can make reliable contact without disturbing the contact of adjacent triads.

### SUMMARY OF THE INVENTION

An improved handheld device is designed for providing laser light treatment to the scalp or other areas of the body benefiting from such treatment. It has been discovered that red and infra red laser light treatments penetrate the skin surface and stimulate growth of hair. Earlier devices intended to take advantage of this discovery suffered from either bulk and expense or from an inability to evenly deliver light treatment in spite of the presence of hair which absorbs or deflects the light. The present device combines a plurality of miniature laser light sources, such as laser diodes, with branched light pipes or direct optical coupling to distribute and deliver the laser light to the scalp surface. When branched light pipes are used, each diode delivers light to multiple points on the scalp surface. With direct optical coupling each laser diode has a lens-like distribution tip. Each individual light pipe or distribution tip acts like the tooth of a comb and moves interfering hairs aside so that the ends of the light pipes or distribution tips physically contacts the skin surface and directly conducts the laser light into the skin.

Not only do the branched light pipes allow each individual laser source to directly deliver light to multiple points on the scalp, use of a trifurcated light pipe results in a stable tripod design for each laser source. Similarly, with direct optical coupling the laser diodes are preferentially arranged in a fixed triad assembly. Just as a stool with three legs can sit evenly on an irregular surface such a tripod or triad allows all the light pipes from a single source or all the distribution tips from a single triad to make contact with the scalp in spite of scalp curvature and individual irregularities. Independently mounting each tripod or triad on a flexible member allows the axis of each tripod or triad to orient independently. This further enhances the ability of the light delivery arrangement to make excellent contact with the scalp surface and deliver and even and repeatable laser light treatments.

A preferred design combines at least four or five laser assemblies (tripods or triads) into a round disc-shaped device that is easily held in the hand. Such a device provides even laser illumination over a circular region with a diameter of about 3 inches (about 7.6 cm). The device includes flexible mounting of the laser assemblies to allow independent orientation and a protective membrane that prevents dirt or debris from penetrating. The device is equipped with a timer so that after the unit is energized and placed on the scalp, a signal is given at an appropriate interval so that the device can be moved to achieve complete and even coverage of the scalp. A storage base is provided so that the ends of the light pipes or distribution tips are protected when the device is not in use. In addition the storage base acts as a charging station to recharge batteries in the handheld unit. The use of rechargeable batteries ensures that the device is completely safe electrically since only low battery voltage is present when the device is in contact with a user's scalp.

### DESCRIPTION OF THE FIGURES

FIG. 1 shows a perspective view of the branched light pipe tripod in relation to a laser diode;

FIG. 2 is shows a perspective view of the branched light pipe tripod engaged with the laser diode;

FIG. 3 shows one design for a molded prism used to couple the laser diode to the branched light pipe tripod;

FIG. 4 shows an alternative design for the molded prism used to couple the laser diode to the branched light pipe tripod;

FIG. 5 shows a diagrammatic cross-section of a branched light pipe illustrating the branching of the laser light beam;

FIG. 6 shows a diagram of the conduction of laser beams within a trifurcated light pipe tripod;

FIG. 7 illustrates the coupling of trifurcated light pipes to a flexible membrane;

FIG. 8 illustrates the branched light pipes making contact with the curved surface of a user's scalp.

FIG. 9 illustrates the use of a second flexible membrane to prevent the entry of dirt into the device;

FIGS. 10A and 10B show possible distribution patterns of trifurcated light pipes or triads;

FIG. 11 is a perspective view of a device incorporating tripods or triads;

FIG. 12 is a side view of the device of FIG. 11;

FIG. 13 shows a perspective view of the device of FIG. 11 showing the light pipes on the device's lower surface;

FIG. 14 is a perspective view of the device of FIG. 11 along with a charging station.

FIG. 15 is a perspective view of a triad assembly where three separate laser diodes are coupled to light distribution tips which assembly can be attached to a flexible membrane in place of the tripod assembly.

FIG. 16 is a diagrammatic view of a cross-section of a light distribution tip showing how the built-in lens focuses the laser light.

### DETAILED DESCRIPTION OF THE INVENTION

The current invention avoids the problems of the prior art by providing a plurality of light pipes or light distribution tips distributed so as to evenly deliver laser radiation to the surface of the scalp. To make efficient use of each laser diode, multiple light pipes can be used to distribute light from each single laser diode. The larger the number of light pipes per laser diode, the lower the amount of radiation coming from any one light pipe due both to the splitting of the primary beam and also to light loss within the light pipe. An optimal number of light pipes for current laser diodes is probably three. This number could be reduced to two or increased to larger numbers particularly with the use of more powerful diodes. However, there are particular advantages to provide three light pipes for each laser diode. Each light pipe acts as a tine or tooth of a comb to bring laser radiation to the scalp free of obstruction by hair, etc., and the tripod configuration allows each light pipe assembly to evenly contact the rounded surface of the scalp. Alternatively, single laser diodes can be optically coupled to light distribution tips, one per laser. To most efficiently interface with the scalp surface such light distribution tips are advantageously arranged in assemblies of threes (triads). Each triad is then able to make contact with a region of the scalp according to the three legged stool principal.

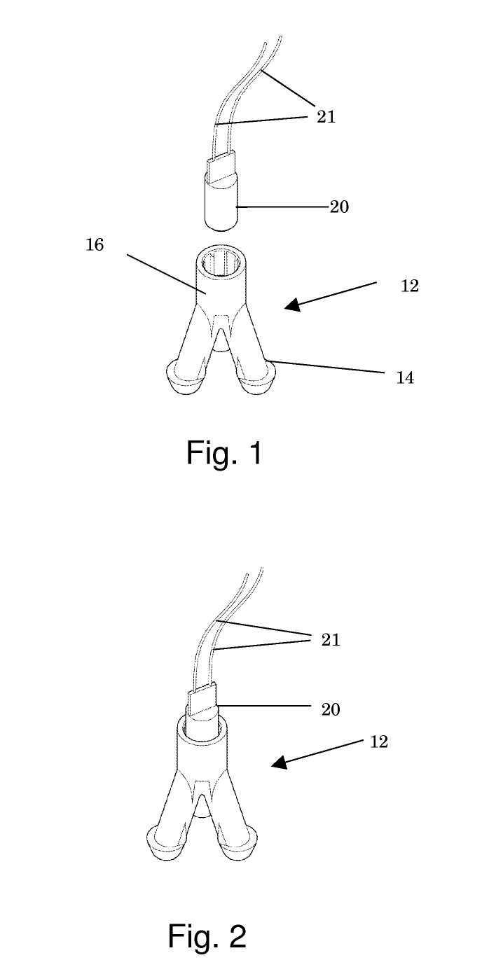

FIGS. 1 and 2 illustrate the concept of connecting a laser (diode) to a light pipe array with multiple (three in this case) branches. FIG. 1 shows the cylindrical laser diode 20 in relation to a branched light pipe tripod 12. The light pipe 12 has an upper region 16 dimensioned to be efficiently optically coupled to the laser diode 20. As is known to one of skill in the art, optical coupling can be achieved by simply bringing the laser diode 20 into optical alignment with the light pipe upper region 16. However, improved results may be obtained by using an optically transmissive agent (such an oil or transparent cement having the proper index of refraction). It will be appreciated that there may be appreciable loss of light within the light pipe 12; however, it is relatively simple to select a laser diode 20 of sufficient power to provide the optimum and safe level of laser radiation at the end of each terminal light pipe 14. In the figure the lower region of the branched light pipe 12 is divided into three separate terminal light pipes or tines 14 equally spaced apart (i.e., at approximately 120 degree intervals) although other numbers of tines could be used and the separation does not necessarily have to be equal, a trifurcation provides certain mechanical advantages—namely a tripod configuration has the advantage of allowing each of the three terminal light pipes 14 of each branched light pipe 12 to contact the surface of the scalp regardless of the scalp's curvature. The branched light pipe 12 is molded or formed from an optically clear plastic such as polystyrene or an acrylic plastic such as polymethacrylate or even from glass. FIG. 2 shows that a single laser beam 18 from the laser diode 20 is divided into three equivalent beams 22 each exiting from one of the light pipes 14.

The figures show the direct coupling of a laser diode to the top surface of the light pipe assembly. One potential problem with such a connection is that there may be suboptimal levels of laser radiation at the end of the tines 14 due to inefficient coupling of the incoming laser beam to the upper region 16 of the light pipe 12. This does not seem to be completely eliminated by optical cements or other approaches to enhancing optical coupling. It appears that the light entering the light pipe may not be at the optimum optical angle or direction for most efficient conduction through the light pipes. As a result light is lost through the walls of the light pipes rather than exiting through the ends of the tines 14 as is desired. One solution to this problem is to mold a “prism” into the top surface of the light pipe. In this case “prism” does not indicate a complete optical prism in which flat surfaces are used to create internal reflections. In the current use “prism” refers to a configuration of flat surfaces which are used to more accurately refract the light from the laser into the individual branches 14 of a branched light pipe 12. Each face or facet 24 of the prism is at approximate right angles to the long axis of its respective light pipe branch so as to optimally conduct light into that branch.

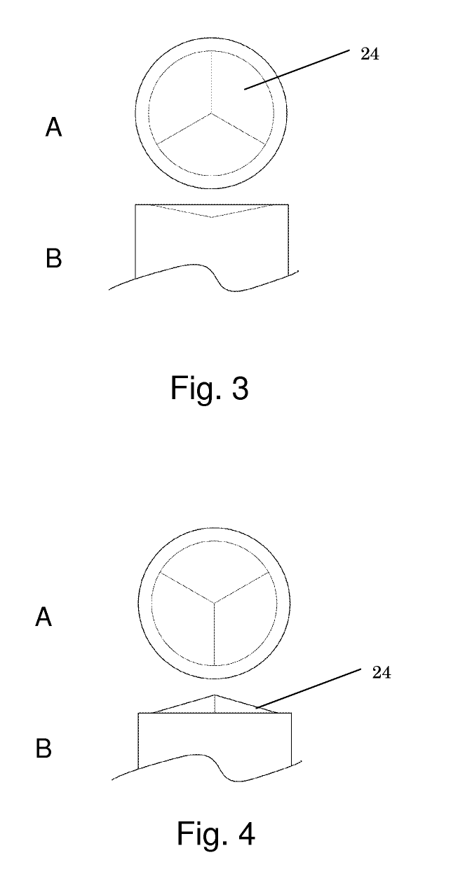

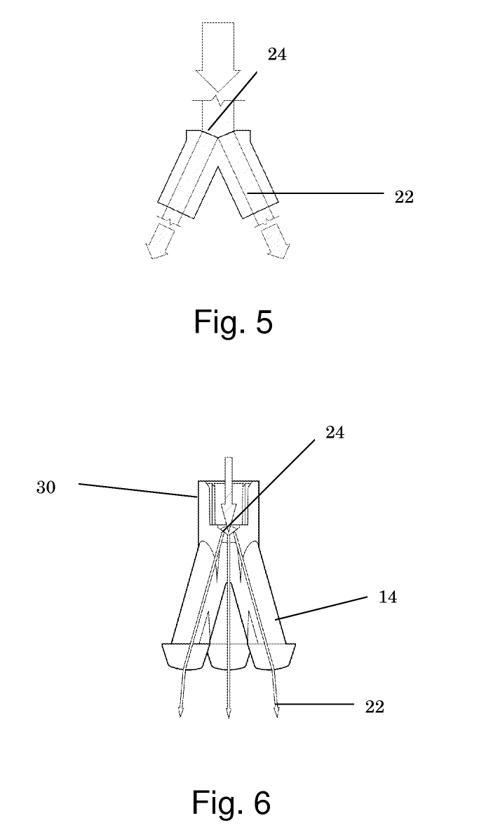

The facets 24 that make up the prism can be either embossed (FIG. 3, top view A, side, view B) (i.e., protruding) or debossed (FIG. 4, top view A, side view B) (i.e., indented). The general configuration is that the number of prism faces 24 is equal to the number of tines or branches 14 in the branched light pipe 12. Essentially, each face 24 is located and angled to maximize the amount of light entering each tine 14 at an angle to ensure optimal transmission as is diagrammed in FIG. 5. Note that in the figure the facets 24 are normal to the axis of each laser beam 22.

As shown in FIG. 6 a recessed collar 30 surrounds the laser so that the light emitting surface of the laser is in close proximity to the faces 24 of the debossed prism. This causes the laser beam to become divided into three direct beams 22—each optimally oriented for transmission by one of the tines 14. To further enhance light transmission the ends 34 of the tines 14 can advantageously be rounded or lens-shaped. The single laser beam from the laser diode 20 is directed towards the center of the prism. Portions of the beam are refracted by each facet thereby directing each portion of the split beam into a separate terminal light pipe or tine 14.

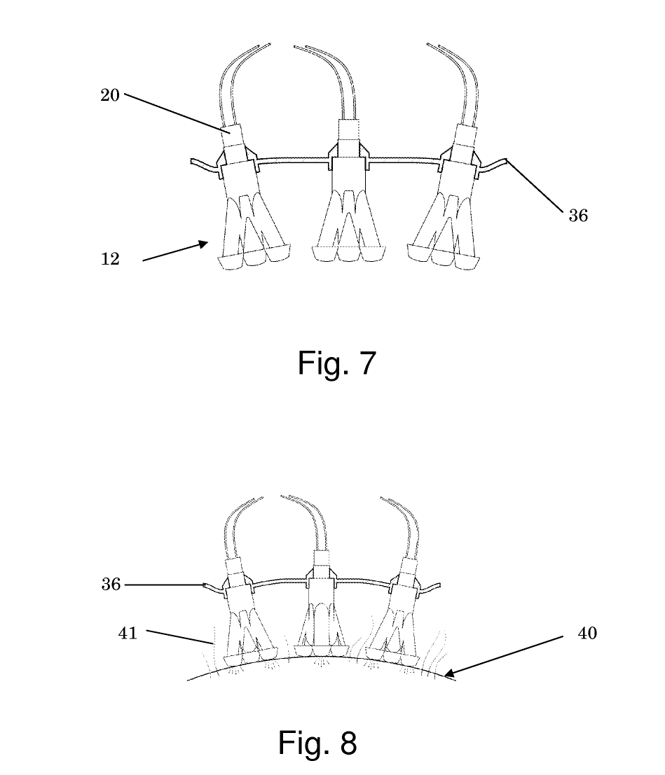

Although it is possible to attach the lasers to a flat (or more likely curved) rigid member, FIG. 7 shows a further improvement in coupling the laser radiation to the scalp can be achieved by attaching the lasers 20 to a flexible member 36 (such as a sheet or band of neoprene, polyurethane or some other resilient elastomer—alternately a separate flexible member attached each laser diode 20 to the device). This arrangement allows the laser diodes 20 and their attached branched light pipes 12 to reorient in response to changes in the geometry of any surface that is contacted. That is, each tripod will naturally make contact with a flat or curved surface. However, if the surface has irregularities such as ridges, adjacent tripods may not all be able to accommodate themselves to the surface if the axis of the laser is fixed. If, however, the laser is attached to a flexible member 36, each laser 20 can individually reorient so that its light pipes make full contact with the surface. In the FIG. 8 the curved surface 40 represents the surface of a user's scalp (with hair 41) receiving light radiation from the device. This same effect can also be achieved by molding the branched light pipes 14 out of an elastomer so that the light pipes themselves flex to ensure contact with the surface of the scalp.

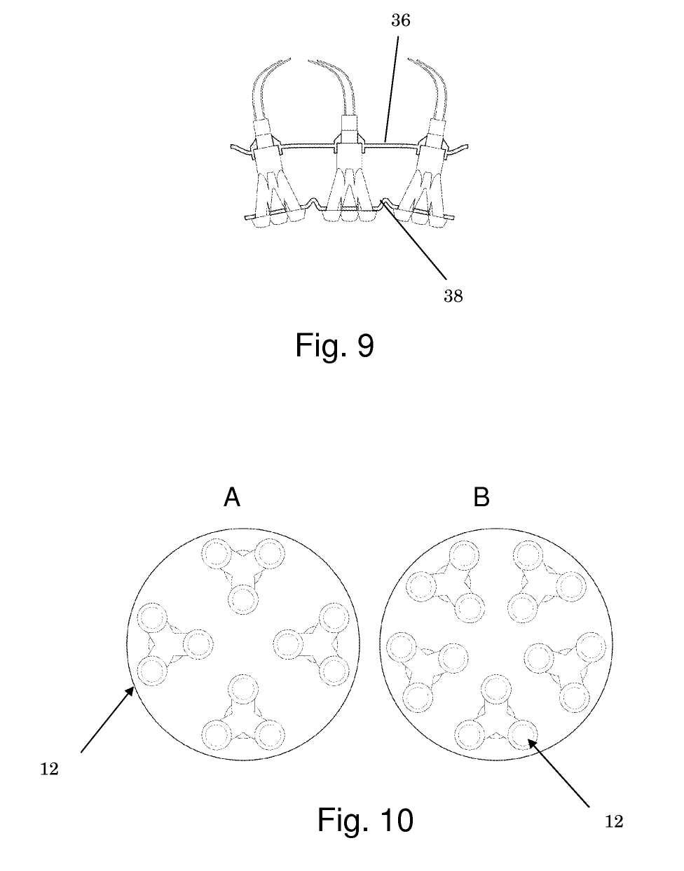

As shown in FIG. 9 an additional improvement can be achieved by providing a sealing membrane 38. The sealing membrane 38 is quite close to the ends of the light pipes 14 which come into contact with the scalp. The tips of each light pipe 14 pass snugly or sealingly through openings in the sealing membrane 38. This arrangement makes the device easier to clean because hair and other debris cannot become tangled around the bodies of the light pipes. Only relatively short blunt ends of the pipes protrude through the sealing membrane so that the whole arrangement can be easily cleaned with a sponge or a soft cloth. Alternatively, sealing membrane can be represented by a rigid layer with the light pipes passing slidingly through openings in the layer. In such an embodiment there is sufficient free space in the openings to allow the light pipes to move relative to the rigid layer; however, the openings are sufficiently small so as to restrict the access of dirt and debris. Other variations such as an o-ring or similar elastic material surrounds each light pipe passing through an opening so as to further restrict entry of contaminants will be apparent to one of skill in the art.

FIG. 10 shows a view of the branched light pipes 12 as seen from the scalp surface. The figure shows that a small number (four in FIG. 10A and five in FIG. 10B) of laser diodes can be spaced apart in a circular pattern to give even coverage of a portion of the scalp. If the circle is sufficiently large and if the density of the diodes is sufficiently high, essentially all regions on the scalp can be simultaneously irradiated. In most practical applications, the circle will be smaller (about three inches or about 7.6 cm in diameter) and/or the density will be lower than that necessary to achieve simultaneous irradiation of the entire scalp surface. Therefore, the device needs to be moved about on the scalp surface to achieve even irradiation. Because the adaptive flexible membrane 36 allows the light pipe ends to automatically accommodate to the scalp surface, the device is easy to maintain in one position on the scalp and easy to move to a second position on the scalp. Such ease of repositioning makes it relatively simple to attain even treatment of the entire scalp.

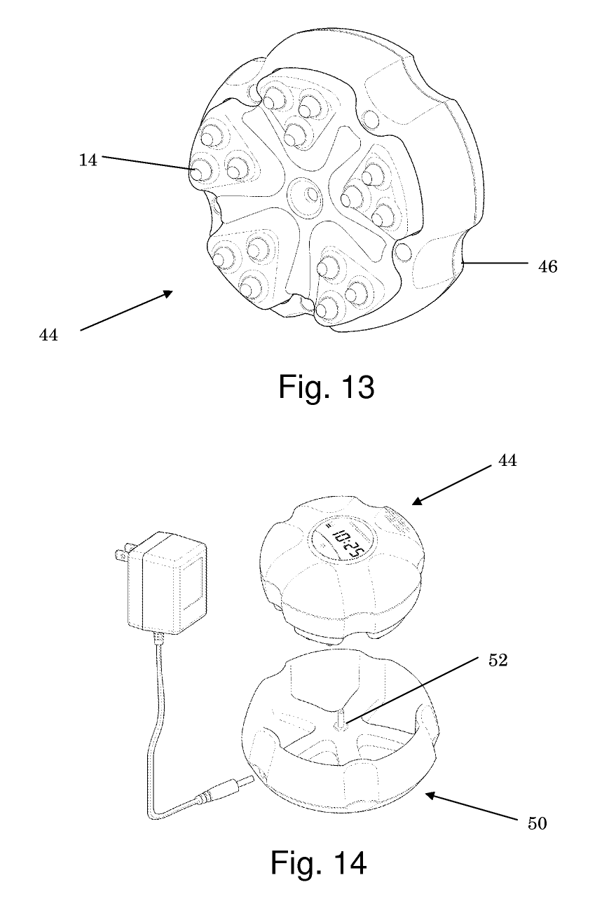

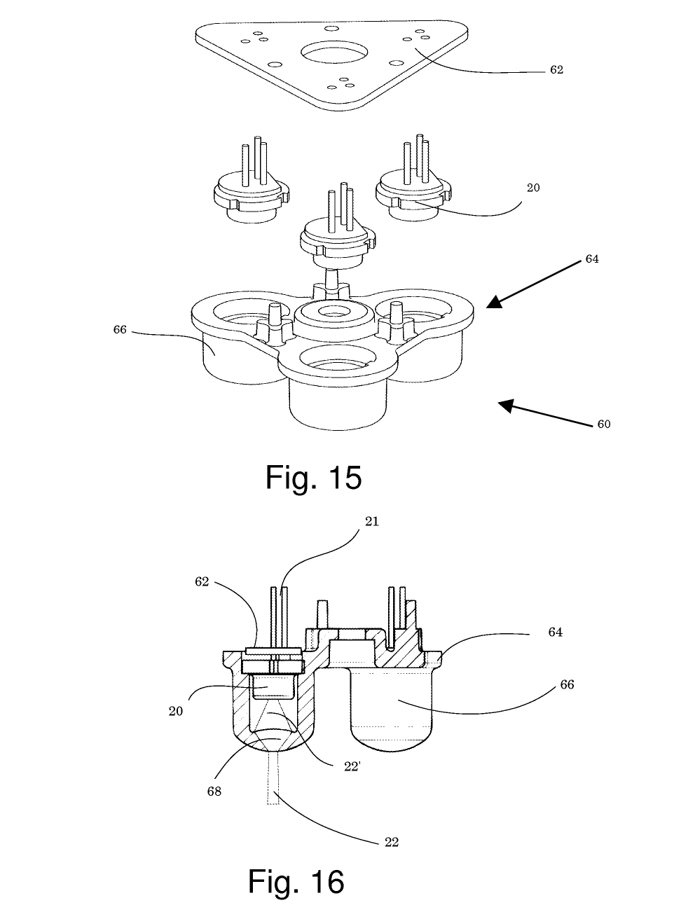

In some cases optimal treatment requires a somewhat higher level of laser light than can be conveniently delivered with the branched light pipe tripod approach discussed immediately above. One simple solution to this problem is to couple individual laser diodes 20 to a light distribution tip 66—one diode per tip. This can advantageously be achieved using a triad assembly 60 as is shown in FIG. 15. The assembly consists of three light distribution tips 66 molded into an assembly 64 so that the tips 66 each end in the same plane. Each light distribution tip 66 is hollow and sized to receive a laser diode 20. A backing plate 62 fixes the diodes 20 in place and is advantageously in the form of a circuit board to which the electrical leads 21 of the diodes 20 can be soldered. Alternatively, the electrical connections 21 can be separate from the backing plate 62. FIG. 16 shows a diagrammatic cross-section through one of the light distribution tips 66 to illustrate how a molded in lens component 68 in the distal end of the tip can focus the laser light 22′ emerging from the diode 20 into a tight beam 22 for optimal interaction with the scalp. A triad assembly 64 of this structure can advantageously be combined with a flexible membrane 36 so that each tripod shown above (FIG. 8) is replaced by a triad. This has the same contact advantages as already explained and can have the same distribution patterns as shown in FIG. 13. The only difference being that each point 14 represents a distribution tip 66 linked to a separate laser diode so that assuming the same strength laser diodes are used in both embodiments, the light distribution tip 66 of a triad delivers approximately three time as much laser light as the light pipe tip of a tripod 12.

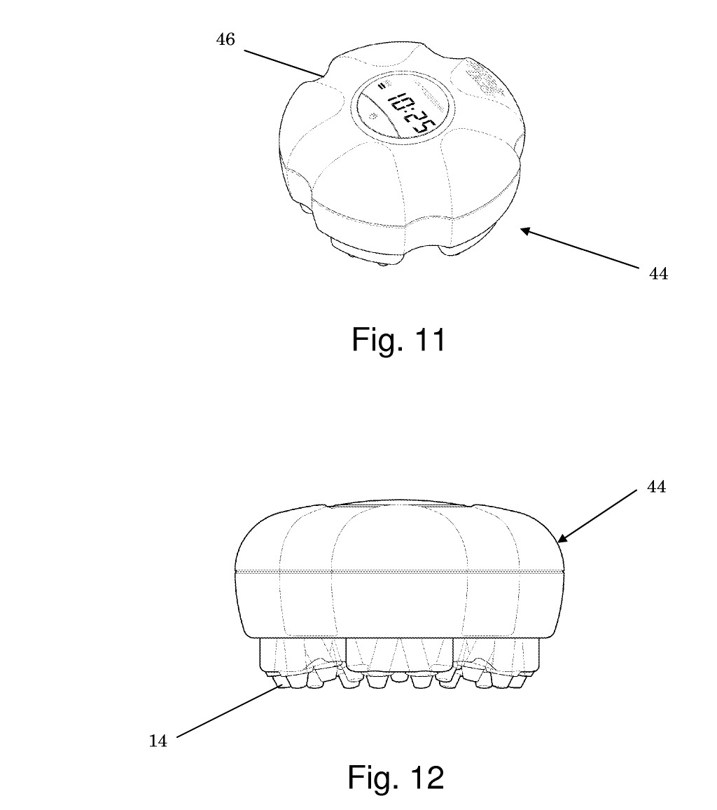

The goal of achieving even treatment is favored by relatively low profile circular device that is shaped so as to be easily grasped. FIG. 11 shows such a domed circular device 44 with circumferential indentations 46 at approximately 72 degree intervals (five indentations) to facilitate grasping the unit. The unit is a convenient size for grasping, preferably between about three and about five inches in diameter. This device is equipped with a lower flexible membrane through which the ends of light pipes 14 or light distribution tips 66 protrude. The laser diodes are located in the region of the device body between the indentations. Although relatively flat in profile, there is sufficient room within the body of the device for the necessary electronics. FIG. 12 shows a drawing of the device 44 in side view illustrating the protrusion of the light pipes 14 or light distribution tips. FIG. 13 shows the device 44 from the lower surface allowing one to see that one branched light pipe (three protruding tines 14) or triad (three light distribution tips 66) is present in each of the five segments defined by the indentations 46. The unit is preferably powered by rechargeable batteries. FIG. 14 shows a storage stand 50 for the device 44 which serves two purposes: 1) it protects the light pipes 14 or light distribution tips 66 when the unit is not in use; and 2) it provides and electrical connection 52 so that the unit automatically recharges while sitting in the storage stand 50.

The unit 44 is used by removing it from the storage stand 50 and activating the laser diodes. The unit is then placed on the scalp and moved about at intervals to ensure even laser light exposure to the region to be treated. The device may be equipped with an integral timer and display to indicate duration of treatment. Periodic sounds (beeps, etc.), lights, tactile ticks or other clues may be used to indicate when the device 44 should be moved. Preferably at the end of the treatment cycle the device automatically turns off to avoid leaving it on by accident.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

We claim:

1. A handheld device for applying laser light to the scalp comprising:
a housing containing an electronic circuit and a power source for the electronic circuit;
a plurality of miniature electronically powered laser light sources electronically connected to the electronic circuit;
a branched light pipe optically coupled to each of said laser light sources, each branch of each branched light pipe positioned so as to contact the surface of the scalp and to conduct laser light directly to the surface of the scalp whereby laser light is applied to the scalp.

2. The device according to claim 1, wherein the number of miniature electronically powered laser light sources is selected from the group consisting of four and five.

3. The device according to claim 1, wherein each branched light pipe comprises three branches.

4. The device according to claim 1, wherein each branched light pipe comprises an integral prism to refract laser light into each of the branches.

5. The device according to claim 4, wherein each integral prism comprises a structure selected from the group consisting of embossed faces and debossed faces.

6. The device according to claim 1, wherein distal ends of the branches of each branched light pipe are rounded.

7. The device according to claim 1 further comprising a flexible member to which is mechanically coupled the miniature electronically powered laser light sources so that each miniature electronically powered laser light source with its optically coupled branched light source can be independently reoriented.

8. The device according to claim 1 further comprising a sealing membrane disposed in proximity to the distal end of each branch of the branched light pipe to prevent contamination of the proximal ends of the branched light pipes

9. A handheld device for applying laser light to the scalp comprising:
a housing containing an electronic circuit and a power source for the electronic circuit;
a plurality of miniature electronically powered laser light sources electronically coupled to the electronic circuit;
a flexible member to which is mechanically coupled the miniature electronically powered laser light sources so that each miniature electronically powered laser light source can be independently reoriented; and
a three branched light pipe optically coupled to each of said laser light sources, each branch of each branched light pipe positioned so as to contact the surface of the scalp and to conduct laser light to the surface of the scalp whereby laser light is applied directly to the scalp.

10. The device according to claim 9, wherein the number of miniature electronically powered laser light sources is selected from the group consisting of four and five.

11. The device according to claim 9, wherein each branched light pipe comprises an integral prism to refract laser light into each of the branches.

12. The device according to claim 11, wherein each integral prism comprises a structure selected from the group consisting of embossed faces and debossed faces.

13. The device according to claim 9, wherein distal ends of the branches of each branched light pipe are rounded.

14. The device according to claim 9 further comprising a sealing membrane disposed in proximity to the distal end of each branch of the branched light pipe to prevent contamination of the proximal ends of the branched light pipes

15. A handheld device for applying laser light to the scalp comprising:
a housing containing an electronic circuit and a power source for the electronic circuit;
a plurality of miniature electronically powered laser light sources electronically coupled to the electronic circuit, each laser light source optically coupled to a light distribution tip which light distribution tip is disposed with two additional light distribution tips to form a planar triad;
a flexible member to which is mechanically coupled the planar triads so that each planar triad can be independently reoriented to allow each light distribution tip of the reoriented planar triad to contact the surface of the scalp and to conduct laser light to the surface of the scalp whereby laser light is applied directly to the scalp.
